(19) United States
(12) Patent Application Publication
(10) Pub. No.: US 2005/0244987 A1
(43) Pub. Date: Nov. 3, 2005

(54) Detection device, detection apparatus, and detection kit employing the device and reagent

(75) Inventors: Norihiko Utsunomiya, Tokyo (JP); Kazuhiro Ban, Tokyo (JP); Takashi Ikeda, Yokohama-shi (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 11/114,073

(22) Filed: Apr. 26, 2005

(30) Foreign Application Priority Data
Apr. 28, 2004 (JP) 2004-134448

(51) Int. Cl. C12Q 1/68; G01N 27/74; G01R 33/12; G01N 33/551; G01N 33/553

(52) U.S. Cl. 436/524; 324/204

(57) ABSTRACT

A detection device for detecting a target substance in a specimen comprises a flow channel for delivering the specimen, a coil surrounding the flow channel, and a reaction region being placed in a portion of the flow channel surrounded by the coil outside and holding a first trap substance immobilized thereon for trapping the target substance, the coil bring about a change of a physical property thereof when the target substance is trapped by the first trap substance and a magnetic body having a second trap substance on the surface thereof which second trap substance is capable of specifically binding to the target substance is trapped by the target substance.

### BACKGROUND OF THE INVENTION

1. Field of the Invention

The present invention relates to a detection device for detecting a target substance in an inspection specimen, a detection apparatus therefor, and a kit employing the element and a reagent.

2. Related Background Art

In recent years, with increase of interest on health problems, environment problems, and safety, micro-detection methods are needed for detecting biological or chemical substances relating to the above problems.

However, in most cases, the size of the specimens containing the objective substance to be detected (hereinafter referred to occasionally as a “target substance”) is limited, and the target substance coexists in a micro quantity complicatedly with various substances. Therefore, the detection method should have high accuracy (reproducibility) and high sensitivity as well as high reliability of the detection.

The clinical detecting apparatus for human health inspection is desirably capable of outputting the detection result in a shorter time after the preparation of the inspection specimen (shortening a so-called turn-around-time) in the practical use of the detecting apparatus. Further for use in a clinical inspection, the detection apparatus should be easily handleable.

Many immunoassay methods have been disclosed which utilize an antigen-antibody reaction of a target substance. Among the disclosed immunoassay methods, for ease of the handling, various bead-methods are disclosed which employ magnetic fine particles holding on the surface thereof an antibody capable of binding specifically to a target substance. In connection therewith, an electrochemical luminescence method by use of magnetic particles is disclosed (U.S. Pat. No. 5,147,806), in which a magnetic property of fine particles is utilized for collection only of the fine particles.

On the other hand, detection by use of fine magnetic particles by utilizing properties of a magnetic material is disclosed in which fine magnetic particles are fixed on the bottom of a reaction cell by an antigen-antibody reaction, and inductance change of an external coil is detected (Anal. Chem. 2004 Mar. 15; 76(6): 1715-9)

Japanese Patent Application Laid-Open No. 2002-286594 discloses a liquid delivery device in which a coil winds around a flow channel. This disclosure is not directed to magnetic fine particles for detection of a target substance in the liquid.

As mentioned above, various detection devices and detection apparatuses are being developed for detection of micro quantities of target substances. Desirably, these apparatuses have high sensitivity for detecting the micro quantity of the target substance, and further are easy in handling and capable of outputting the detection results in a shorter time.

### SUMMARY OF THE INVENTION

The present invention enables detection of an antigen or antibody by immunoassay by utilizing an antigen-antibody reaction, or detection of a nucleic acid by hybridization of a complementary nucleic acid sequence, with easily handleable apparatus in a shorter time for data output with high detection sensitivity.

According to an aspect of the present invention, there is provided a detection device for detecting a target substance in a specimen, comprising:
a flow channel for delivering the specimen,
a coil surrounding the flow channel, and
a reaction region being placed in a portion of the flow channel surrounded by the coil outside and holding a first trap substance immobilized thereon for trapping the target substance,
the coil bring about a change of a physical property thereof when the target substance is trapped by the first trap substance and a magnetic body having a second trap substance on the surface thereof which second trap substance is capable of specifically binding to the target substance is trapped by the target substance.

The device preferably further comprises additionally a sensor element for sensing the change of a physical property.

The change of a physical property is preferably a change of a magnetic flux density in a magnetic field formed by the coil.

Alternatively, the change of a physical property is preferably a change of an inductance of the coil.

The coil is preferably placed in plurality, and the sensor element is provided between the coils.

The reaction region preferably holds a porous material or a fine structure.

According to another aspect of the present invention, there is provided an apparatus for detecting a target substance in a specimen, comprising:
an assembly holding the above detection device, and
a sensor means which senses the change of a physical property bought about by the coil when the target substance is trapped by the first trap substance and a magnetic body having a second trap substance on the surface thereof is trapped by the target substance.

According to a further aspect of the present invention, there is provided a kit for detecting a target substance in a specimen, comprising:
a detection device comprising
a flow channel for delivering the specimen,
a coil surrounding the flow channel and
a reaction region placed in a portion of the flow channel surrounded by the coil outside; and
a detection reagent containing magnetic fine particles having on a surface thereof a trapping substance capable of binding specifically to the target substance.

In the present invention, a coil is placed around a reaction region in a flow channel. With this coil placement, a physical change induced in the coil is detected with high sensitivity in comparison with placement of the coil out of the reaction region. Further, use of a porous material or fine structure, which has a large surface area per unit volume, for constituting the reaction region improves the reaction efficiency and enables shortening of the detection time. Furthermore, a magnetic label used for the detection does not deteriorate with time, and variation among the production lots is less in comparison with labels of biological origins such as enzymes and fluorescence. Thereby, the detection device, detection apparatus, and detection kit are readily handleable.

### BRIEF DESCRIPTION OF THE DRAWINGS

FIG. 1 illustrates schematically an device of the present invention.

FIG. 2 illustrates schematically another device of the present invention.

FIGS. 3A and 3B illustrate schematically still another device of the present invention.

FIG. 4 illustrates schematically the device employed in Example 2 of the present invention.

FIG. 5 illustrates schematically the device employed in Example 2 of the present invention.

FIG. 6 illustrates schematically a complex formed in the reaction region of the present invention.

FIGS. 7A, 7B, 7C, 7D, 7E, 7F, 7G, 7H and 7I illustrate schematically porous materials and fine structures for constituting the reaction region of the present invention.

FIG. 8 is a diagram of the oscillation circuit employed in Example 2 of the present invention.

### DETAILED DESCRIPTION OF THE PREFERRED INVENTION

The detection device of the present invention is an device for detecting a target substance in a specimen. The detection device comprises a flow channel for delivering the specimen; a coil surrounding the flow channel; a reaction region being placed in a portion of the flow channel surrounded by the coil outside and holding a first trap substance immobilized therein for trapping the target substance. The coil changes a physical property thereof when the target substance is trapped by the first trap substance and a magnetic body having a second trap substance on the surface thereof is trapped by the target substance.

The reaction region is constituted preferably from a porous material or a fine structure for a larger surface area.

In the present invention, when plural reaction regions are formed in the flow channel, preferably a coil is provided for each of the reaction regions, and the plural reaction regions are arranged such that the magnetic fields generated by the coils are brought on common lines.

In the present invention, when the flow channel is formed in a ring shape, the coil is preferably provided to wind around the ring-shaped flow channel and to bring the magnetic field in the same line at the inlet end and outlet end of the flow channel.

For sensing a change of the magnetic flux density of the coil, the sensor element for sensing the magnetic flux density is preferably placed at the end portion of the coil on an imaginary plane perpendicular approximately to the magnetic field generated by the coil. The sensor element for sensing the magnetic flux density may be provided inside the flow channel, or outside the flow channel on an imaginary plane perpendicular approximately to the magnetic field generated by the coil.

When plural coils are provided and the magnetic flux density sensor element is provided in the flow channel, the magnetic flux density sensor is preferably placed between the coils in the flow channel.

When the flow channel is formed in a ring shape, the magnetic flux density sensor element is preferably placed at the flow inlet end or at the flow outlet end in the flow channel, or between the flow inlet end and the flow outlet end.

The detection apparatus of the present invention comprises an assembly holding the detection device of the present invention, and a sensing means which senses the change of a physical property caused in the coil when the target substance is trapped by the first trap substance and a magnetic body having a second trap substance on the surface thereof is trapped by the target substance.

The change of the physical property is preferably a change of the magnetic flux density of the coil or a change of an inductance of the coil. The change of the inductance of the coil can be measured by a change of the oscillating frequency. For the measurement, the coil is connected to an oscillating circuit, and the oscillation frequency of the oscillation circuit is counted which varies depending on the change of the inductance. The oscillating circuit is preferably an LC oscillation circuit.

The present invention further provides a kit for detecting a target substance in a specimen, comprising a detection device comprising a flow channel for delivering the specimen; a coil surrounding the flow channel; and a reaction region placed in a portion of the flow channel surrounded by the coil outside; and a detection reagent containing magnetic fine particles having on a surface thereof a trapping substance capable of binding specifically to the target substance. In this kit, the magnetic flux density sensor element may be provided in the flow channel.

Embodiments of the present invention are described below by reference to drawings.

(Detection Device)

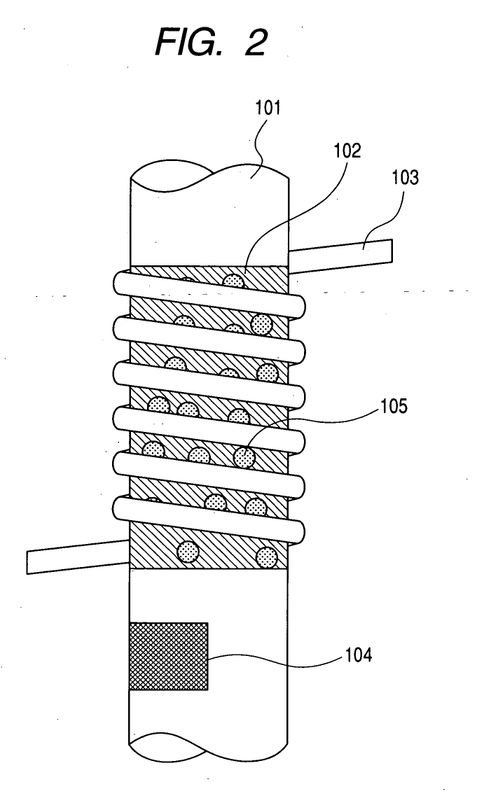

An embodiment of the present invention is described by reference to FIG. 2.

Flow channel 101 in this embodiment is a capillary tube. Otherwise, the flow channel may be a groove formed on a chip.

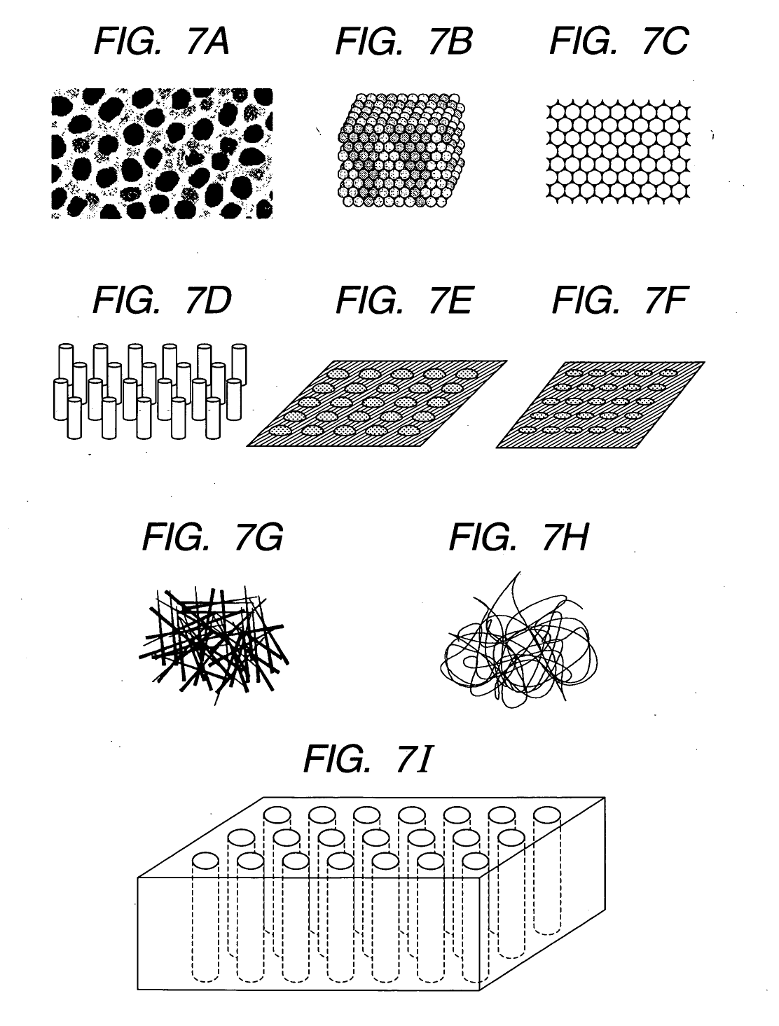

In flow channel 101, a porous material or fine structure is provided as reaction region 102. Examples of the porous material and fine structure include a pore structure (FIG. 7A), an opal structure (FIG. 7B), a reversed opal structure (FIG. 7C), a fine particle aggregation structure (irregular arrangement of fine particles of an opal structure, not shown in the drawing), a column structure (FIG. 7D), a protrusion structure (FIG. 7E), a depression structure (FIG. 7F), a projection structure (FIG. 7G), a fiber structure (FIG. 7H), and a hollow structure (FIG. 7I).

A trap substance which is capable of forming selectively a binding pair with a target substance is immobilized on the surface of the porous material. The trap substance for trapping the target substance is not specially limited, provided that the trap substance is capable of forming a bonding pair specifically with the target substance. The target substances contained in the inspection specimen are classified roughly into biological substances and non-biological substances.

The non-biological substances of a high industrial value include environmental pollutants such as PCBs of various chlorine substitution numbers and positions, and dioxins of various chlorine substitution numbers and positions; incretion-disturbing substances called environmental hormones such as hexachlorobenzene, pentachlorophenol, 2,4,5-trichlorophenoxyacetic acid, 2,4-dichlorophenoxyacetic acid, amitorol, atrazine, arachlor, hexachlorocyclohexane, ethylparathion, chlordane, oxychlordane, nonachlor, 1,2-dibromo-3-chloropropane, DDT, kelthane, aldrin, endrin, dieldrin, endosulfane (benzoepin), heptachlor, heptachlor epoxide, malathion, mesomil, methoxychlor, mylex, nitrophene, toxaphene, triflurarin, alkylphenols (5-9 carbons), nonylphenol, octylnonylphenol, 4-octylphenol, bisphenol-A, di-2-ethylhexyl phthalate, butyl benzyl phthalate, di-n-butyl phthalate, dicyclohexyl phthalate, diethyl phthalate, benz(a)pyrene, 2,4-dichlorophenol, di-2-ethylhexyl adipate, benzophenone, 4-nitrotoluene, octachlorostyrene, aldicarb, benomil, kiepone (chlordecon), manzeb (mancozeb), maneb, methylam, metribdin, dipermethrin, esfenvalerate, fenvalerate, permethrin, binchlozorin, zineb, ziram, dipentyl phthalate, dihexyl phthalate, and dipropyl phthalate.

The biological substances include nucleic acids, proteins, sugar chains, lipids, and complexes thereof. Specifically, the biological substances include biological molecules selected from nucleic acids, proteins, sugar chains, and lipids. More specifically, the present invention can be applied to substances containing any of DNAs, RNAs, aptamers, genes, chromosomes cell membranes, viruses, antigens, antibodies, lectins, haptens, hormones, receptors, enzymes, peptides, sphingosugars, and sphingolipids. Further, bacteria or cells which produce the above biological substance can be a target substance of the object of the present invention.

Specific examples of the proteins are so-called disease markers.

Examples of the disease marker include:
α-fetoprotein (AFP) as an acidic glycoprotein that is produced in the liver cells during the fetus period and is present in the fetal blood, and as a marker for hepatocellular carcinoma (primary liver cancer), hepatoblastoma, metastatic liver cancer, and a yolk sac tumor;
PIVKA-II as an abnormal prothrombin appearing in the case of hepatocyte dysfunction, which is confirmed to appear specifically in hepatocellular carcinoma;
BCA225 as a glycoprotein that is immunohistochemically a breast cancer-specific antigen, and as a marker for primary and advanced breast cancer, recurrent breast cancer, and metastatic breast cancer;
basic fetoprotein (BFP) as a basic fetoprotein discovered in a serum, intestine tissue extract and brain tissue extract of the human fetus, and as a marker for ovarian cancer, a testic tumor, prostate cancer, pancreatic cancer, biliary tract cancer, hepatocellular carcinoma, renal cancer, lung cancer, stomach cancer, bladder cancer, and colon cancer;
CA15-3 as a carbohydrate antigen which is a marker for advanced breast cancer, recurrent breast cancer, primary breast cancer, and ovarian cancer;
CA19-9 as a carbohydrate antigen which is a marker for pancreatic cancer, biliary tract cancer, stomach cancer, liver cancer, colon cancer, and ovarian cancer;
CA72-4 as a carbohydrate antigen which is a marker for ovatian cancer, breast cancer, colorectal cancer, stomach cancer, and pancreatic cancer;
CA125 as a carbohydrate antigen which is a marker for ovarian cancer (in particular, serous cystadenocarcinoma), adenocarcinoma of the corpus uteri, allopian tube cancer, adenocarcinoma of the uterine cervix, pancreatic cancer, lung cancer, and colon cancer;
CA130 as a glycoprotein which is a marker for epithelial ovarian cancer, fallopian tube cancer, lung cancer, hepatocellular carcinoma, and pancreatic cancer;
CA602 as a core protein antigen which is a marker for ovarian cancer (in particular, serous cystadenocarcinoma), adenocarcinoma of the corpus uteri and adenocarcinoma of the uterine cervix;
CA54/61 (CA546) as a nuclear matrix sugar chain-related antigen which is a marker for ovarian cancer (in particular, muscinous cystadonocarcinoma), adenocarcinoma of the uterine cervix, and adenocarcinoma of the corpus uteri;
carcinoembryonic antigen (CEA) which is now most widely used for assisting cancer diagnosis as a marker antigen related to tumors such as colon cancer, stomach cancer, rectal cancer, biliary tract cancer, pancreatic cancer, lung cancer, breast cancer, uterine cancer, and urinary system cancer;
DUPAN-2 as a carbohydrate antigen which is a marker for pancreatic cancer, biliary tract cancer, hepatocellular carcinoma, stomach cancer, ovarian canacer, and colon cancer;
eleastase-1 as a pancreatic exocrine protease which exists in the pancreas and specifically hydrolyzes an elastic fiber, elastin, of the connective tissue (which constitutes the artery wall, tendon or the like), and as a marker for pancreatic cancer, pancreatic cystic adenocarcinoma, and biliary tract cancer;
immunosuppressive acidic protein (IAP) as a glycoprotein that exists in high concentration in the ascites or serum of the human cancer patient, and as a marker for lung cancer, leukemia, esophageal cancer, pancreatic cancer, ovarian cancer, renal cancer, bile duct cancer, stomach cancer, bladder cancer, colon cancer, thyroid cancer, and malignant lymphoma;
NCC-ST-439 as a carbohydrate antigen which is a marker for pancreatic cancer, biliary tract cancer, breast cancer, colon cancer, hepatocellualr carcinoma, lung adenocarcinoma, and stomach cancer;
γ-seminoprotein (γ-Sm) as glycoprotein which is a marker for prostate cancer;
prostate-specific antigen (PSA) as a glycoprotein extracted from the human prostate tissue, which exists in the prostate tissue and is therefore a marker for prostate cancer;
prostate acidic phosphatase (PAP) as an enzyme secreted from the prostate and hydrolyzing a phosphoric ester under acidic pH conditions, which is used as a tumor marker for prostate cancer;
nerve-specific enolase (NSE) as a glycolytic enzyme that exists specifically in the nerve tissue and neuroendocrine cells, and as a marker for lung cancer (in particular, lung small cell cancer), neuroblastoma, a nerve tumor, pancreas islet cancer, esophagus small cell cancer, stomach cancer, renal cancer, and breast cancer;
squamous cell carcinoma-related antigen (SCC antigen) as a protein extracted and purified from the liver metastatic focus of uterine cervix squamous cell carcinoma, which is a marker for uterine cancer (cervix squamous cell carcinoma), lung cancer, esophageal cancer, head and neck cancer, and skin cancer;
sialyl Le^{X}-I antigen (SLX) as a carbohydrate antigen which is a marker for lung adenocarcinoma, esophageal cancer, stomach cancer, colon cancer, rectal cancer, pancreatic cancer, ovarian cancer, and uterine cancer;
SPan-1 as a carbohydrate antigen which is a marker for pancreatic cancer, biliary tract cancer, liver cancer, stomach cancer, and colon cancer;
tissue polypeptide antigen (TPA) as a marker for esophageal cancer, stomach cancer, colorectal cancer, breast cancer, hepatocellular carcinoma, biliary tract cancer, pancreatic cancer, lung cancer, and uterine cancer, which is a single-stranded polypeptide that identifies progressive cancer in combination with other tumor markers, in particular, and is useful for relapse prediction, and treatment follow-up;
sialyl Tn andigen (STN) as a nuclear matrix carbohydrate antigen, which is a marker for ovarian cancer, metastatic ovarian cancer, stomach cancer, colon cancer, biliary tract cancer, pancreatic cancer, and lung cancer;
CYFRA (cytokeratin) as a tumor marker effective for detecting lung non-small cell cancer, in particular lung squamous cell carcinoma;
pepsinogen (PG) as an inactive precursor for two pepsines (PGI, PGII) that are protein digestive enzymes secreted into the gastric juice, and as a marker for gastric ulcer (in particular, lower gastric ulcer), duodenal ulcer (in particular, recurrent and intractable duodenal ulcers), Brunner's gland adenoma, Zollinger-Ellison syndrome, and acute gastritis;
C-reactive protein (CRP) as an acute phase response protein that mutates by a tissue disorder or infection, of which the level is high when myocardial necrosis occurs due to acute myocardial infarction or the like;
serum amyloid A protein (SAA) as an acute phase response protein that mutates by a tissue disorder or infection;
myoglobin as a hemoprotein with a molecular weight of about 17,500 that exists mainly in the myocardium and skeletal muscle, which is a marker for acute myocardial infarction, myodystrophy, polymyositis, and dermatomyositis;
BNP as a cerebral diuretic peptide constituted of 32 amino acids originating in a ventricle, which is useful for diagnosing a disease state of cardiac incompetence;
ANP similarly as a diuretic peptide originating in an atrium cordis;
creatine kinase (CK) (three kinds of isozyme of CK-MM derived from skeletal muscle, CK-BB derived from brain or smooth muscle and CK-MB derived from myocardium, and CKs (macro CKs) that bind to mitochondrial isozyme or immunoglobulin) as an enzyme that exists mainly in the soluble fraction of skeletal muscle and myocardium and is released into the blood by cell injury, which is a marker for acute myocardial infarction, hypothyroidism, progressive myodystrophy, and polymyositis;
troponin T as a protein with a molecular weight of 39,000 that forms a troponin complex together with troponin I, C on a thin filament of striated muscle and is involved in the regulation of muscle contraction, which is a marker for rhabdomyolysis, myocarditis, myocardial infarction and renal failure;
cardiac myosin light chain I as a protein contained in the cells of skeletal muscle and the cells of myocardium, which is a marker for acute myocardial infarction, myodystrophy, and renal failure because the results in which increase of the measured value indicates dysfunction or necrosis of skeletal muscle or myocardium; and
chromogranin A, thioredoxin, 8-OHdG, and cortisol that have attracted attention as stress markers in recent year.

The antibody, an example of the trap substance, herein signifies immunoglobulins which are produced in a living body in the natural world, or synthesized entirely or partially by a genetic engineering technique, a protein engineering technique, or an organic reaction. Further, the antibody in the present invention includes all derivatives of the above immunoglobulins which retain the specific binding properties. Furthermore, the antibody includes proteins which have a binding domain highly homologous to the binding domain of the immunoglobulin (including chimerical antibodies and humanized antibodies). The antibody or immunoglobulin is produced in a living body in the natural world, or synthesized or modified entirely or partially.

The antibody or the immunoglobulin may be a monoclonal antibody or a polyclonal antibody specific to a target substance.

The antibody or the immunoglobulin may be any member of immunoglobulin classes including the human classes (IgG, IgM, IgA, IgD, and IgE). In the present invention, derivatives of IgG class are preferable.

The term “antibody fragment” signifies any molecule or complex shorter than the full length of an antibody or an immunoglobulin. The antibody fragment retains preferably the main portion of the specific binding ability of the entire length of the antibody.

The antibody fragment is exemplified by Fab, Fab′, F(ab′)2, scFv, Fv, diabodies, and Fd fragments, but is not limited thereto.

The antibody fragment may be produced by any method. For example, an antibody fragment may be produced by fragmenting an intact antibody enzymatically or chemically, or produced by genetic engineering from a gene coding for a partial antibody sequence. Otherwise, the antibody fragment can be produced entirely or synthesized partially. The antibody fragment may be produced in a single-stranded antibody fragment as necessary. The fragment may contain plural strands connected by a disulfide (S—S) linkage. The fragment may be a complex of plural molecules. A functional antibody fragment contains typically about 50 amino acids, more typically about 200 amino acids.

The term “variable domain” in the present invention signifies a front portion of an immunoglobulin having an amino acid sequence portion which differs with antigens for function of the specific binding/trapping to the target substance (antigen), being usually referred to as Fv.

Fv is constituted of a variable domain of a heavy chain (hereinafter referred to occasionally as “VH”) and a variable domain of a light chain (hereinafter referred to occasionally as “VL”). The immunoglobulin G contains usually two VH domains and two VL domains respectively.

In the present invention, the functional portion of the variable domain of the heavy chain or light chain of the immunoglobulin (hereinafter occasionally referred to simply as a “functional portion”) has actually specificity to a target substance (antigen). The term, functional portion, is used to denote a portion called academically CDR (complementary determining region), and, in particular, a portion having actually specificity to a target substance (antigen).

The interaction between the target substance and the trap substance may be any interaction, provided that a physical/chemical change by the binding can be detected by the detection device of the present invention. Preferred interaction includes an antigen-antibody reaction, an interaction between an antigen and an aptamer (RNA fragment having a specified structure), an interaction between a ligand and receptor, DNA hybridization, an interaction between DNA and a protein (transcription factor, etc.), and an interaction between a lectin and a sugar chain.

In FIG. 2, coil 103 is formed to wind around the reaction region. Below coil 103, sensor element 104 is placed for sensing a magnetic flux density.

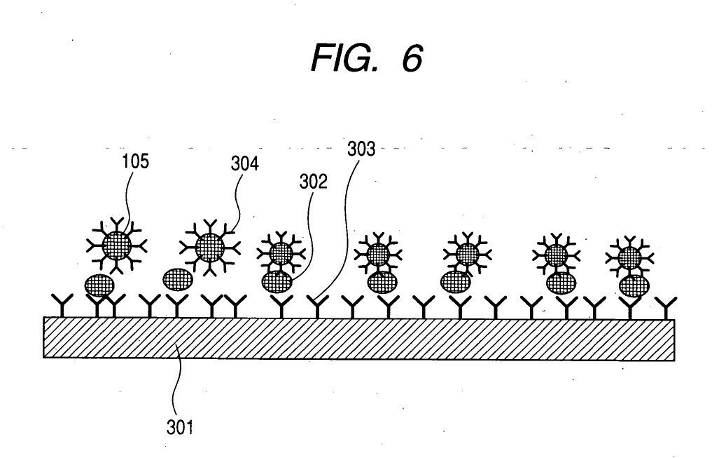

Various types of sensor elements are useful, including hole elements, MR elements, flux gate elements, MI elements, and TMF elements. The aforementioned trap substance (magnetic-particle-immobilized antigen) 304 for catching a target-substance is immobilized on the surface of magnetic particles 105 (see FIG. 6).

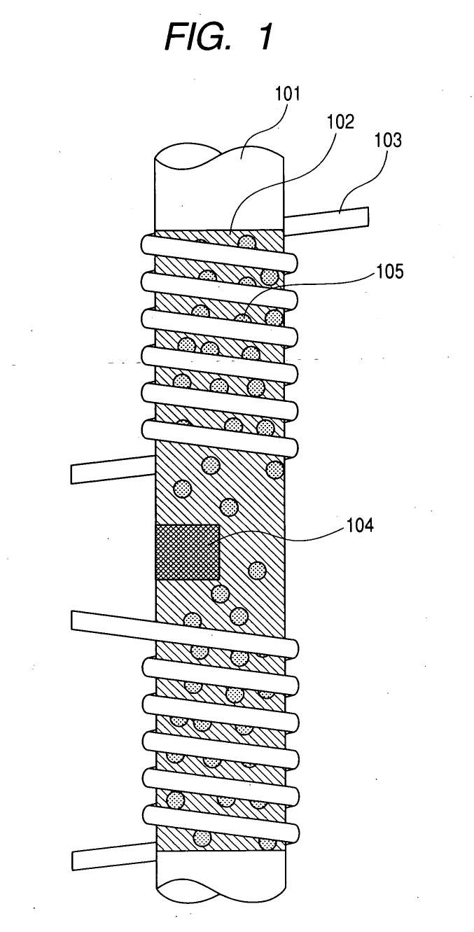

In reaction region 102 in FIG. 1, fixing antibody 303 is immobilized on the reaction region formed on the surface of the porous material or fine structure constituting reaction region 102, and target substance-trap substance (antibody immobilized on the magnetic fine particles) 304 is immobilized on magnetic fine particles 105: target substance 302 is immobilized between fixing antibody 303 and target substance-trap substance 304.

Magnetic fine particles 105 are made preferably of a ferromagnetic material such as iron, cobalt, and nickel, and alloy thereof, and have preferably superparamagnetic properties. Generally, a ferromagnetic material particles having a diameter of several tens to several hundreds of nanometers or less come to be magnetically saturated at a weak magnetic field, but has superparamegnetic properties without hysteresis and without residual magnetization. The size of magnetic fine particles 101 is preferably in the range in which the particles have superparamagnetic properties. When the fine particles are mainly made of a polymer with superparamagnetic fine particles dispersed therein, the particle size can be made to be larger than several hundreds of nanometers.

In the detection procedure, a constant electric current is allowed to flow through coil 103, and magnetic flux density of the magnetic field formed by coil 103 is measured by magnetic density sensor element 104. The magnetic flux density depends on the quantity of magnetic fine particles 105 immobilized in the coil-surrounded region. The quantity of the target substance in the inspection specimen can be derived from the magnetic flux density by comparison with a calibration curve preliminarily prepared by use of solutions of known concentrations of the target substance.

### EXAMPLES

The present invention is explained below by reference to drawings without limiting the invention in any way.

### First Example

A first example is explained by reference to drawings.

(Preparation of Device)

A monolithic capillary column, MonoCap (G.L. Science Co.), is useful as the flow channel device shown in FIG. 1. This capillary column holds a porous silica material of a three dimensional structure immobilized in the capillary.

The surface of the silica was treated with 3-glycidoxypropyltrimethoxysilane to provide epoxy groups thereon. Into the capillary column, a solution of an antihuman insulin monoclonal antibody was introduced, and was immobilized on the silica surface. The column was washed with a phosphate buffer solution. Most of the antibody was immobilized with the antigen-recognition sites kept free, since the antibody has many amino groups reactive highly to the epoxy groups on the Fc site thereof.

Around the above-prepared capillary containing the immobilized antibody, coil 103 was provided as shown in FIG. 1. Two sets of capillaries of this constitution were prepared. The two capillaries were bonded with interposition of a capillary having a magnetic flux density sensor element 104 sealed therein. The bonding of the capillaries is preferably conducted by a method other than thermal fusion not to damage the function of the immobilized antibody: specifically, by bonding with an adhesive, or by a joining member for joining the capillaries.

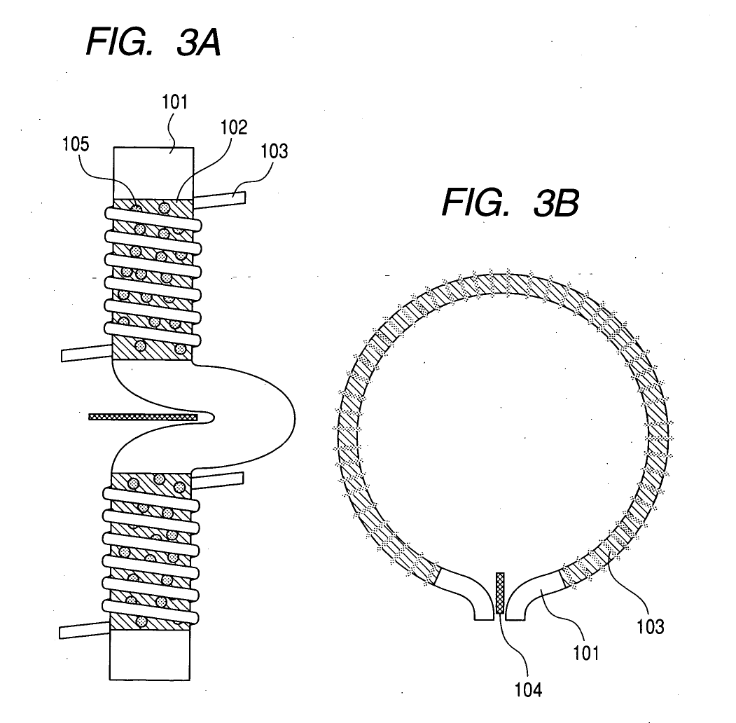

In this example, two coils were counterposed. However, a single coil constitution as shown in FIG. 2 is also useful. Further, in this example, the flow channel had a linear shape, and the detection device is placed inside the flow channel. However, the detection device may be placed outside the flow channel as shown in the constitution in FIGS. 3A and 3B.

FIG. 3A shows a constitution in which magnetic flux density sensor 104 is placed directly under the coil and the flow channel is formed so as to avoid magnetic flux density sensor element 104.

FIG. 3B shows a constitution in which the flow channel is in a shape of a circular arc and the coil is provided in a toroidal shape outside the flow channel, and magnetic flux density sensor element 104 is placed at the interspace between the inlet and outlet of the flow channel. With this constitution, one coil forms a circular magnetic circuit. Incidentally, in the constitution of FIG. 3B, magnetic flux density sensor 104 may be provided in the flow channel at the inlet end or outlet end thereof.

In the constitution shown in FIG. 3A or 3B, the magnetic flux density sensor element is placed outside the flow channel, which reduces the cost of the detection apparatus.

In the constitution shown in FIG. 1 or FIG. 3A in which magnetic flux density sensor element 104 is placed between the two coils, the magnetic fields generated by the coils should be directed in the same direction relative to magnetic flux density sensor element 104. In the constitution shown in FIG. 1 or FIG. 3A, the coil above magnetic flux density sensor element 104 and the coil below magnetic flux density sensor element 104 are wound in reversed directions, or the direction of the electric current flow is reversed with the two coils wound in the same direction.

With the two coils placed above and below magnetic flux density sensor element 104 as shown in FIG. 1 or FIG. 3A, the magnetic force lines are formed to pass though the two coils without expansion outside, which enables measurement of the magnetic flux density at a high density and high sensitivity.

(Detection Reagent)

The reagent 105 which is constituted of magnetic fine particles and a target substance-trapping agent immobilized thereon as shown in FIG. 6 is prepared as described below. In the examples, this magnetic fine particulate material used is Dynabeads M-270 Epoxy (Dynal Biotech Co.) constituted of polystyrene beads of average particle size of 2.8 μm in which Fe₂O₃ is distributed. This bead-containing liquid and an antihuman insulin monoclonal antibody solution are mixed and incubated overnight. The antihuman insulin monoclonal antibody is preferably capable of recognizing a binding site different from that of the antibody immobilized on the capillary. After the incubation, the magnetic fine particles are caught by a magnet and washed with a phosphate buffer solution containing 0.1% Tween 20 (surfactant). After washing, the particles are released from the magnet and are dispersed by stirring in a phosphate buffer solution.

(Detection Method)

The steps of detection with the device described above are explained below.

An inspection specimen solution containing a target substance to be detected is mixed preliminarily with the above detection reagent. The mixture is introduced into the capillary explained above by reference to FIG. 1. After the introduction, the capillary is washed with a phosphate buffer solution containing 0.1% Tween 20 (surfactant). Thereby, as explained above by reference to FIG. 6, the target substance 302 is immobilized between reaction-region-fixed antibody 303 formed on the porous material or a fine structure constituting reaction region 102 and a target substance-trapping substance (magnetic fine particle-fixed antibody) 304 immobilized on the surface of magnetic fine particles 105.

In this state, a constant electric current is allowed to flow to generate magnetic fields in the two coils 103 in the same direction. The density of the magnetic flux formed by the coils is measured by magnetic flux density sensor element 104.

The measured magnetic flux density is a function of the quantity of magnetic fine particles 105 immobilized in the coils. For measurement of the quantity of the target substance, a calibration curve is prepared preliminarily with insulin solutions of known concentrations to derive the relation between the concentration and the measured magnetic flux density in the same manner as described above. The quantity of the target substance is determined from the magnetic flux density caused by the solution of an unknown concentration.

In the above steps, the specimen and the reagent are preliminarily mixed, and then the mixture solution is introduced into the detection device. In another method, the specimen solution may be firstly introduced into the detection device and washed, and then the reagent solution may be introduced therein. However, for detection of a monomer having one binding site of one kind like insulin is preferably conducted by the former method. In contrast, a multimer having plural binding sites of one kind like C-reactive protein is preferably detected by the latter method to avoid aggregation of the magnetic beads.

### Second Example

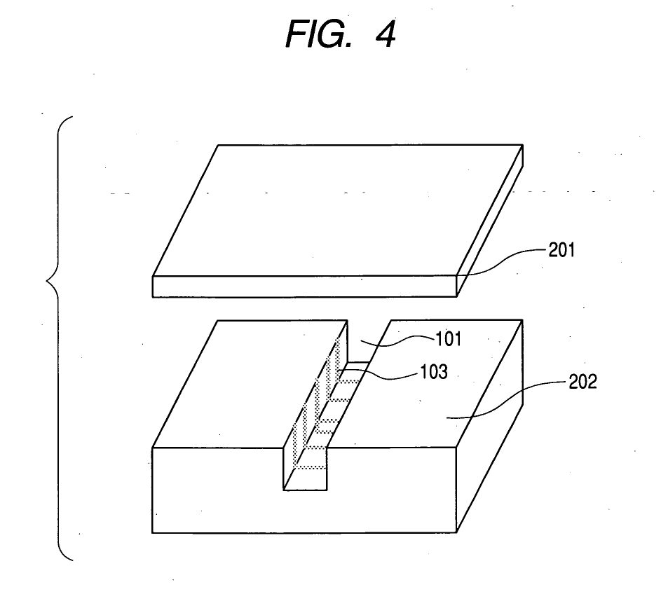

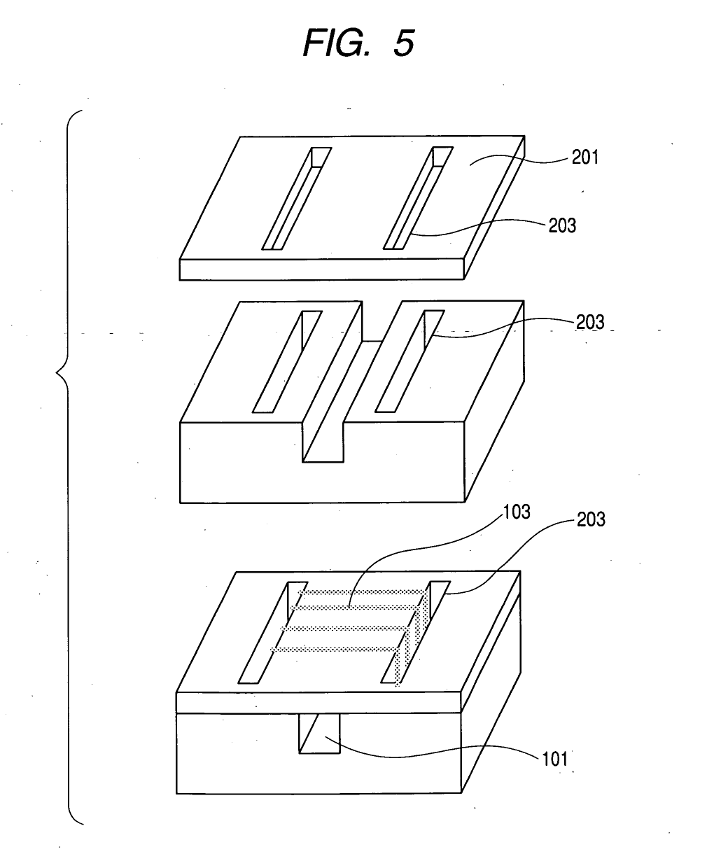

Second Example is explained below by reference to FIGS. 4 and 5.

(Constitution of Detection Device)

The flow channel device shown in FIG. 4 is in a shape of a chip employing a μ-fluidics. FIG. 4 illustrates schematically the reaction region cut out from the device.

Top cover 201 and base plate 202 of the chip are made of heat-resistant glass in this embodiment. Flow channel 101 on base plate 202 is formed by grooving the base plate 202. Portions of coil 103 are formed in flow channel 101, and other portions of this coil 103 are formed also on a face of top cover 201 of the chip (the coil portions formed on the back face of the top cover 201 being not shown). By fitting the top cover 201 to the chip base plate 202, the portions of coil 103 formed on flow channel 101 and the portions of coil 103 formed on top cover 201 are connected to complete coil 103.

The portions of coil 103 formed in flow channel 101 and the portions of the coil 103 formed on top cover 201 can be formed from gold (Au) by a lift-off process. After formation of the portions of coil 103 on flow channel 101 and on top cover 201, the top cover and the base plate are joined to complete the chip.

In the same manner as in the above first Example, an antihuman insulin antibody is immobilized on flow channel 101. The immobilization of the antihuman insulin antibody is conducted by treating the surface of the gold coil with a thiol having an NHS group, and allowing a solution of the antihuman insulin antibody to flow for the immobilization.

Another example of the detection device on the chip is shown in FIG. 5. In this embodiment, slits 203 are formed in chip top cover 201 and chip base plate 202 respectively in the direction parallel to the reaction region in the flow channel. Slits 203 formed in chip top cover 201 and chip base plate 202 are made to penetrate through the base plate. Flow channel groove 101 is formed on base plate 202.

Wiring lines for forming the coil 103 are formed on chip top cover 201, on the reverse face of the bottom of chip base plate 202, and on the side faces of slits of chip top cover 201 and of chip base plate 202.

Flow channel 101 is formed by working the base plate 202. In this embodiment, the material of the base plate is PDMS (polydimethylsiloxane). The reaction region of the flow channel is treated by oxygen plasma, and is immediately treated with 3-glycidoxypropyltrimethoxysilane to provide epoxy groups on the surface. Then a solution of antihuman insulin monochronal antibody is introduced into the flow channel to immobilize the antibody.

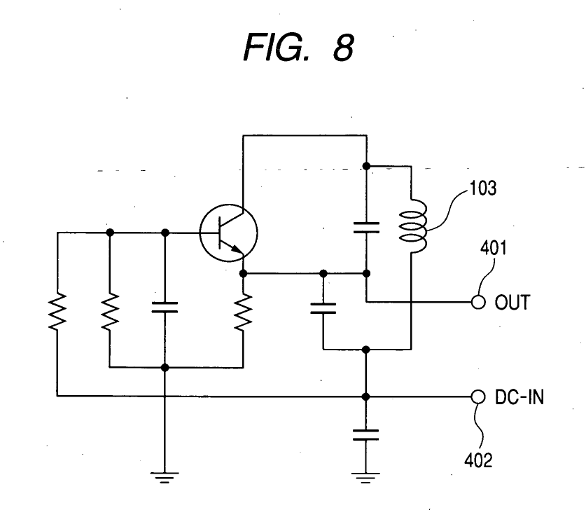

The device as shown in FIG. 4 or FIG. 5 is connected to an LC oscillation circuit like the one shown in FIG. 8 to employ the coil as a part of the oscillation circuit.

(Detection Regent)

The detection reagent in the first example is also useful in this embodiment.

(Detection Steps)

The steps of the detection with the above-described device are explained below. A solution of a specimen containing a target substance and the above detection reagent are preliminarily mixed. The mixture solution is introduced into the chip shown in FIG. 4 or FIG. 5. Then a phosphate buffer solution containing 0.1% Tween 20 (surfactant) is introduced for washing. Thereby the complex is formed on the surface of the reaction region of the flow channel as shown in FIG. 6. In this state, a constant voltage is applied to terminal 402 of the oscillation circuit shown in FIG. 8 from a DC source (not shown in the drawing).

The oscillating frequency outputted from terminal 401 of the oscillation circuit shown in FIG. 8 is counted by a frequency counter. Since the oscillating frequency of this oscillating circuit depends on the coil inductance, the oscillating frequency varies in correspondence with the quantity of the trapped magnetic fine particles.

For determining the concentration of a target substance from the measured oscillating frequency, a calibration curve is prepared preliminarily at plural known concentrations of the insulin solution through the same steps as the measurement of the target substance to obtain the relation between the concentration and the oscillating frequency. The concentration of the target substance in the specimen solution can be determined from the oscillating frequency of the specimen solution.

The oscillating circuit is not limited to the one shown in FIG. 8, and may be any LC oscillating circuit capable of changing the oscillating frequency in correspondence with a coil reactance and a condenser capacity.

In the present invention, the coil is wound around the reaction region of the flow channel in which the magnetic fine particles are immobilized, whereby the physical change caused in the coil can be sensed with strong response, or with high sensitivity, in comparison with case where the coil is placed out of the reaction region. Further in the present invention, the porous material or fine structure in the reaction region has a large surface area per unit volume, which improves the reaction efficiency and enables shortening of the detection time. Furthermore, the magnetic label used for the detection does not deteriorate with time, and variation between production lots is less, which renders the handling easier.

This application claims priority from Japanese Patent Application No. 2004-134448 filed Apr. 28, 2004, which is hereby incorporated by reference herein.

1. A detection device for detecting a target substance in a specimen, comprising:
a flow channel for delivering the specimen,
a coil surrounding the flow channel, and
a reaction region being placed in a portion of the flow channel surrounded by the coil outside and holding a first trap substance immobilized thereon for trapping the target substance,
the coil bring about a change of a physical property thereof when the target substance is trapped by the first trap substance and a magnetic body having a second trap substance on the surface thereof which second trap substance is capable of specifically binding to the target substance is trapped by the target substance.

2. The detection device according to claim 1, wherein the device further comprises additionally a sensor element for sensing the change of a physical property.

3. The detection device according to claim 2, wherein the change of a physical property is a change of a magnetic flux density in a magnetic field formed by the coil.

4. The detection device according to claim 2, wherein the change of a physical property is a change of an inductance of the coil.

5. The detection device according to claim 2, wherein the coil is placed in plurality, and the sensor element is provided between the coils.

6. The detection device according to claim 1, wherein the change of a physical property is a change of a magnetic flux density in a magnetic field formed by the coil.

7. The detection device according to claim 1, wherein the change of a physical property is a change of an inductance of the coil.

8. The detection device according to claim 1, wherein the reaction region holds a porous material or a fine structure.

9. An apparatus for detecting a target substance in a specimen, comprising:
an assembly holding the detection device set forth in claim 1, and
a sensor means which senses the change of a physical property bought about by the coil when the target substance is trapped by the first trap substance and a magnetic body having a second trap substance on the surface thereof is trapped by the target substance.

10. A kit for detecting a target substance in a specimen, comprising:
a detection device comprising a flow channel for delivering the specimen,
a coil surrounding the flow channel and
a reaction region placed in a portion of the flow channel surrounded by the coil outside; and
a detection reagent containing magnetic fine particles having on a surface thereof a trapping substance capable of binding specifically to the target substance.
